(19) United States
(12) Patent Application Publication
(10) Pub. No.: US 2008/0319495 A1
(43) Pub. Date: Dec. 25, 2008

(54) External Defibrillator Having a Ceramic Storage Capacitor and Energy Conditioning Circuit

(75) Inventors: Steven C. Hugh, Bothell, WA (US); Daniel J. Powers, Issaquah, WA (US)

Correspondence Address: PHILIPS MEDICAL SYSTEMS;PHILIPS INTELLECTUAL PROPERTY & STANDARDS, P.O. BOX 3003, 22100 BOTHELL EVERETT HIGHWAY, BOTHELL, WA, 98041-3003, US

(73) Assignee: KONINKLIJKE PHILIPS ELECTRONICS N.V., EINDHOVEN (NL)

(21) Appl. No.: 11/575,152

(22) PCT Filed: Sep. 12, 2005
(86) PCT No.: PCT/IB2005/052982

§ 371 Date: Mar. 13, 2007

Related U.S. Application Data
(60) Provisional application No. 60/611,331, filed on Sep. 20, 2004.

(51) Int. Cl. A61N 1/04

(52) U.S. Cl. 607/5

(57) ABSTRACT

An external defibrillator for providing a defibrillating pulse to a patient includes a ceramic storage capacitor an d an energy conditioning circuit. A charging circuit coupled to the ceramic storage capacitor electrically charges the ceramic storage capacitor, which has an electrical discharge characteristic. The energy conditioning circuit coupled to the ceramic storage capacitor receives the electrical energy discharging according to the electrical discharge characteristic, and in response, provides output energy according to a modified electrical discharge characteristic. The output energy is delivered by a steering circuit to a patient as a defibrillating pulse having a defibrillating pulse characteristic.

This invention generally relates cardiac defibrillators, and more particularly, to external defibrillators employing high -voltage ceramic capacitors to store the electrical energy that is delivered to a patient.

Sudden cardiac arrest (SCA) most often occurs without warning, striking people with no history of heart problems. It is estimated that more than 1000 people per day are victims of sudden cardiac arrest in the United States alone. SCA results when the electrical component of the heart no longer functions properly causing an abnormal sinus rhythm. One such abnormal sinus rhythm, ventricular fibrillation (VF), is caused by abnormal electrical activity in the heart. As a result, the heart fails to adequately pump blood through the body. VF may be treated by applying an electric shock to a patient's heart through the use of a defibrillator.

Defibrillators include manual defibrillators, automatic or semi -automatic external defibrillators (AEDs), defibrillator/monitor combinations, advisory defibrillators and defibrillator trainers. A defibrillator shock clears the heart of abnormal electrical activity (in a process called “defibrillation”) by producing a momentary asystole and providing an opportunity for the heart's natural pacemaker areas to restore normal rhythmic function. Currently available external defibrillators provide either a monophasic or biphasic electrical pulse to a patient through electrodes applied to the chest. Monophasic defibrillators deliver an electrical pulse of current in one direction, whereas biphasic defibrillators deliver an electrical pulse of current first in one direction and then in the opposite direction. When delivered external to the patient, these electrical pulses are high -voltage, high-energy pulses, typically in excess of 1000 volts and in the range of 100 to 300 Joules of energy.

Of the wide variety of external defibrillators currently available, AEDs are becoming increasingly popular because they can be used by relatively inexperienced personnel. Additionally, these external defibrillators can be made relatively lightweight, compact, and portable, such as those used by paramedics and EMS personnel, or attached to carts such as those found in clinics and hospitals. However, the portability of a defibrillator is limited by hardware and design constraints. For example, with respect to design constraints, conventional design rules for high-energy and high-voltage systems, such as are used in an external defibrillator, dictate that the high-voltage components of the defibrillator be spaced apart by a minimum distance requirement. As a result, the physical size of the defibrillator is affected since the defibrillator case mu st be sufficient to accommodate the minimum spacing design rule.

With respect to hardware constraints, various components of the defibrillator are selected for their stability over a wide range of environmental operating conditions, such as varying temperature and humidity. One such component is a storage capacitor of the defibrillator, which typically is used to store electrical energy that is eventually delivered to a patient as a defibrillating pulse. As previously mentioned, the defibrillating pulses can be in excess of 1000 volts and are typically in the range of hundreds of Joules of energy. Consequently, the storage capacitor of a defibrillator typically has a capacitance between 100 and 200 μF and is rated for approximately 2000 volts. The storage capacitors are further selected based on the ability to maintain stable capacitance characteristics over a wide range of temperatures since the AEDs are deployed over such a wide range of different environments such as those encountered by emergency rescue vehicles in a variety of different climates. Conventional storage capacitors are typically film or electrolytic capacitors that are several cubic inches in volume. The resulting storage capacitors, which have sufficient capacitance and voltage characteristics, as well as suitable stability over various environmental operating conditions, have physical dimensions which constitute a significant portion of an AEDs overall size. As a result, minimizing the overall size of the defibrillator will be limited by the physical dimensions of a typical storage capacitor. Therefore, to facilitate reducing the physical size of an external defibrillator, an alternative design is desirable.

The present invention is directed to an external defibrillator for providing defibrillating pulses to a patient including a ceramic storage capacitor and an energy conditioning circuit. The ceramic storage capacitor has an electrical discharge characteristic and is electrically charged by a charging circuit coupled to the ceramic storage capacitor. The energy conditioning circuit is also coupled to the ceramic storage capacitor and is configured to receive the electrical energy discharging according to the electrical discharge characteristic from the ceramic storage capacitor and, in response, provide the electrical energy according to a modified electrical discharge characteristic. A steering circuit coupled to the energy conditioning circuit is configured to couple the electrical energy discharging according to the modified electrical discharge characteristic to a pair of electrodes to deliver a defibrillating pulse having a defibrillating pulse characteristic to the patient.

### BRIEF DESCRIPTION OF THE DRAWINGS

FIG. 1 is a functional block diagram of an external defibrillator according to an embodiment of the present invention.

FIG. 2 is a functional block diagram of a high-voltage delivery circuit of the external defibrillator of FIG. 1.

FIG. 3 is a functional block diagram of an energy storage circuit of the high-voltage delivery circuit of FIG. 2.

FIG. 4 is a top plan view and a side view of a ceramic storage capacitor of the energy storage circuit of FIG. 3.

FIGS. 5A-5C are schematic drawings of various energy conditioning circuits that can be utilized in the energy storage circuit of FIG. 3.

Embodiments of the present invention are directed to an external defibrillator that includes a ceramic storage capacitor and an energy conditioning circuit for conditioning the discharge o f the ceramic capacitor. The physical size of the resulting external defibrillator can be reduced over conventional external defibrillators since ceramic capacitors are typically more compact for a given capacitance than their film and electrolytic counterparts. Ceramic capacitors, however, are subject to wide variation in capacitance value with temperature changes. This characteristic has limited the use of ceramic capacitors to indwelling defibrillators where the ceramic capacitor temperature can be expected to remain within a few degrees of normal body temperature of 98.6° F. This characteristic has heretofore caused ceramic capacitors to be seen as unacceptable for use in external defibrillators. However, an energy conditioning circuit is included in embodiments of the present invention to accommodate performance shortcomings of ceramic capacitors that have prevented utilization of ceramic capacitors in external defibrillators. In the following description certain specific details are set forth in order to provide a thorough understanding of embodiments of the present invention. It will be clear, however, to one skilled in the art, that the present invention can be practiced without these details. In other instances, well-known circuits have not been shown in detail in order to avoid unnecessarily obscuring the description of the various embodiments of the invention. Also not presented in any great detail are those well -known control signals and signal timing protocols associated with the internal operation of defibrillators.

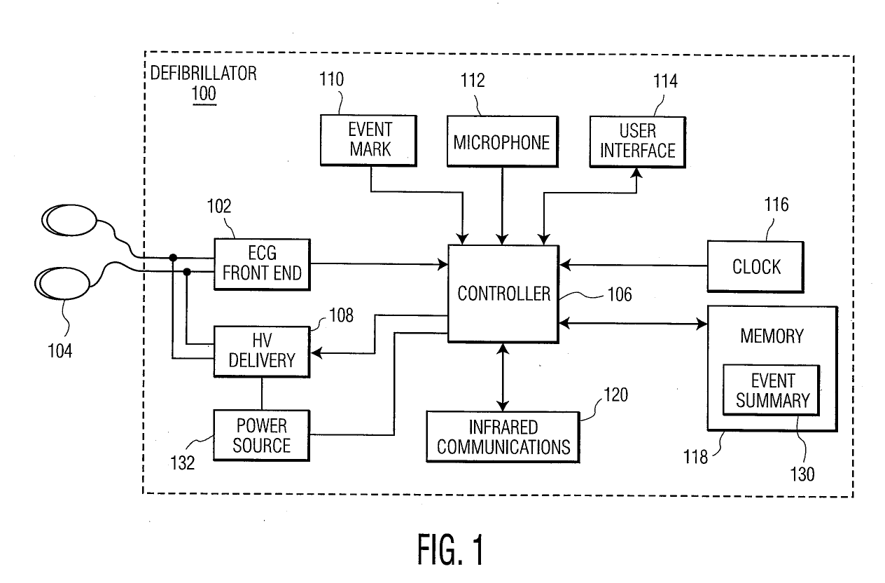

FIG. 1 is a schematic representation of the defibrillator 100. A pair of electrodes 104 are included to provide a defibrillating pulse when attached to a patient. The electrodes 104 further provide an ECG signal to the ECG front end 102, which provides the ECG signal to a controller 106 for evaluation and display to the operator via a user interface 114. The ECG information can also be stored by the controller 106 in a memory 118. The memory 118 can also be used to store an event summary 130, in which information from an event mark 110, a microphone 112, and/or from a clock 116 are stored. The event summary information is useful during a transfer (often called a handoff) between an emergency medical technician and a hospital in order to continue the treatment of the patient. An infrared communications port 120 is also included in the defibrillator 100 to communicate the information in the memory 11 8 with an outside device during the transfer.

A power source 132 included in the defibrillator 100 provides power to the entire defibrillator 100. The power source 132 can be a line source or a battery, or any similar device which provides sufficient power for a defibrillating pulse and the ECG monitoring functions described herein. The power source 132 is typically a disposable or rechargeable battery for portable external defibrillators such as the defibrillator 100. A high voltage (HV) delivery circuit 108 administers a defibrillating pulse to the patient via the electrodes 104 at the command of the controller 106. At the instigation of the operator using a shock button (not shown), the charge from the high voltage delivery device 108 is administered to the patient in order to bring about the normal rhythmic ventricular contractions. The power source 132 supplies the charging energy to the high voltage delivery device 108 during a charging time in order to store sufficient energy to administer a treatment. The charging time is preferably small since the rapid administration of the treatment is desirable to produce a favorable result.

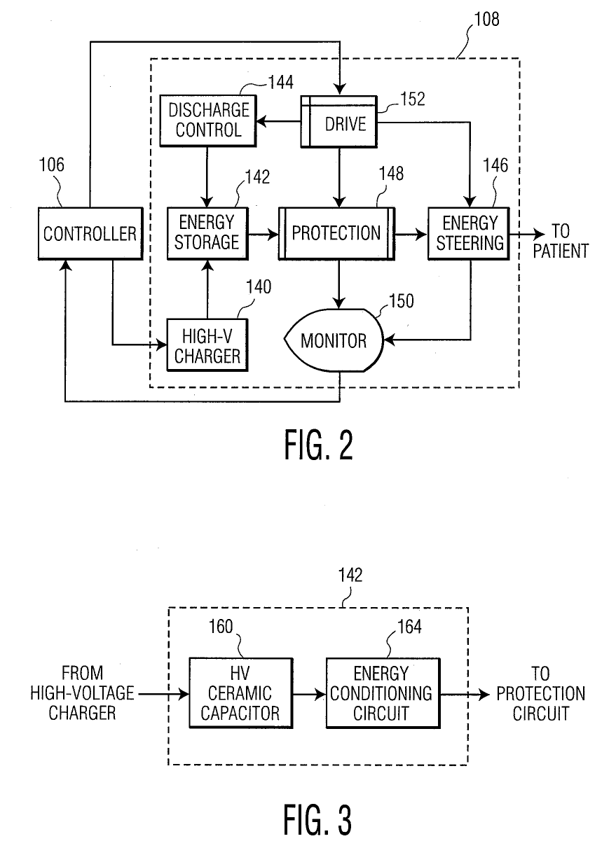

As shown in FIG. 2, the high-voltage delivery circuit 108 includes a number of functional circuit blocks which are both monitored and controlled by the controller 106. A high-voltage charging circuit 140, such as a flyback power supply, responds to one or more control signals issued by the controller 106 and generates electrical energy for provision to an energy storage circuit 142. The storage circuit 142 stores the electrical energy for subsequent delivery to the patient. A discharge control circuit 144 controls discharge of the energy stored in the energy storage circuit 142 to an energy transfer or steering circuit 146 through a protection circuit 148. The steering circuit 146 in turn delivers the electrical energy to the patient via the electrodes 104 (FIG. 1). The steering circuit 146 may deliver the electrical energy to the patient with a single polarity (e.g., a monophasic pulse) or with an alternating polarity (e.g., a biphasic or multiphasic pulse), as required by the desired implementation. In one embodiment of the present invention, the energy steering circuit 146 is has an “H-bridge” configuration, with four switching elements (not shown), such as silicon controlled rectifiers (SCRs). The switching of the four switching elements to deliver a defibrillating pulse, monophasic or biphasic, is under the control of a drive circuit 152.

The protection circuit 148 functions to limit energy delivery from the energy storage circuit 142 to the steering circuit 146 and to discharge or otherwise disarm the energy storage circuit 142 in the event of a fault condition. The protection circuit 148 operates to limit the time-rate-of-change of the current flowing through the steering circuit 146. A monitor circuit 150 senses operations of both the protection circuit 148 and the steering circuit 146 and reports the results of such monitoring to the controller 106. The above-described operations of the discharge control circuit 144, the steering circuit 146, and the protection circuit 148 are controlled by the drive circuit 152 issuing a plurality of drive signals. Operation of the drive circuit 152 is, in turn, controlled by one or more control signals provided by the controller 106.

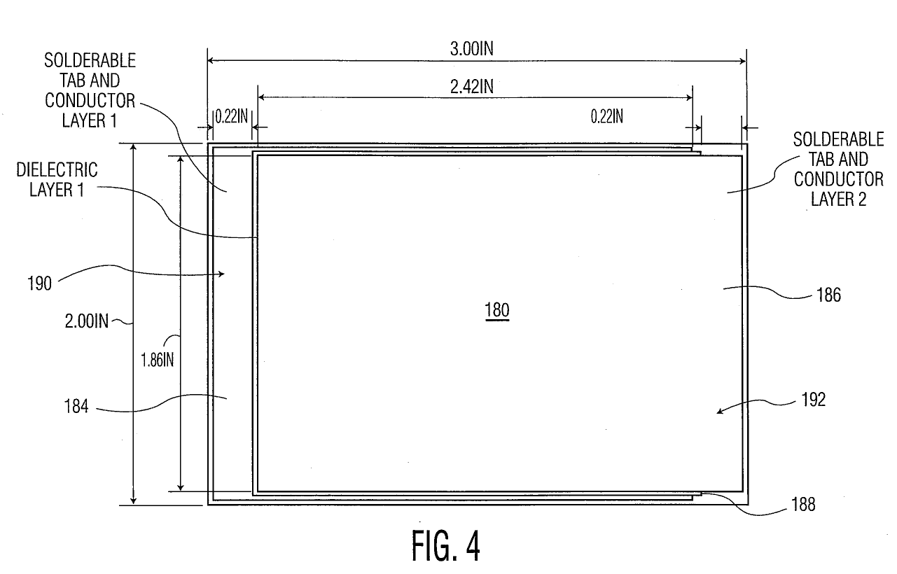

FIG. 3 illustrates the energy storage circuit 142 according to an embodiment of the present invention. The energy storage circuit 142 includes a ceramic storage capacitor 160 coupled to an energy conditioning circuit 164. The ceramic storage capacitor 160 is made from a monolithic construction technique and is formed from a ceramic substrate that is used as a construction base. An example of a ceramic capacitor 180 that can be used for the ceramic storage capacitor 160 is illustrated in FIG. 4. As shown for the embodiment of FIG. 4, the dimensions of the ceramic capacitor 180 are approximately three inches by two inches. The thickness of the ceramic capacitor 180 is approximately one-tenth of an inch. The resulting volume of the ceramic capacitor 180 is less than three-quarters of a cubic inch. The ceramic capacitor 180 is formed from a first conductive layer 184 and a second conductive layer 186, with a dielectric layer 188 disposed between the first and second conductive layers 184, 186. Solderable tabs 190 and 192 are located on opposite sides of the ceramic capacitor 180 to provide a means for electrically connecting the ceramic capacitor 180 to the circuitry of an external defibrillator 100. The solderable tab 190 is formed as an extension of the first conductive layer 184 and the solderable tab 192 is formed as an extension of the second conductive layer 186. Generally, the overlap of the dielectric layer 188 and the conductive layers 184,186 define the capacitive area of the ceramic capacitor 180. Preferably, the dielectric constant of the material from which the dielectric layer 188 is formed has a high K value. Examples of the materials that can be used in forming the dielectric layer 188 include compositions of lead, magnesium lanthanum, zirconium, titanium, and the like. It will be appreciated to one ordinarily skilled in the art, however, that alternative materials currently known, or later developed, can be used as well without departing from the scope of the present invention.

When used in high-voltage applications, such as in external defibrillators, ceramic capacitors exhibit non-linear capacitance characteristics that result in charging and discharging characteristics that are different compared to conventional film or electrolytic capacitors. Additionally, ceramic capacitors lack the stability of conventional film capacitors, especially with respect to temperature. That is, the temperature coefficient of capacitance (TCC) are typically high for ceramic capacitors, causing the capacitance characteristics of ceramic capacitors to vary with temperature. Ceramic capacitors have been employed in implantable defibrillators, as previously mentioned. However, the relatively constant temperature inside the body of a patient, that is, the environment in which the implantable defibrillator operates, mitigates the temperature sensitivity of ceramic capacitors. In contrast to the relatively stable temperature environment of a human body, external defibrillators, especially AEDs, are operated in a variety of temperature conditions. As a result, conventional external defibrillators have not been designed with ceramic capacitors in part due to the difficulties caused by non-linear capacitance characteristics and high TCC.

As previously discussed, the energy storage circuit 142 includes an energy conditioning circuit 164 coupled to the ceramic storage capacitor 160. The energy conditioning circuit 164 conditions the electrical energy discharged from the ceramic storage capacitor 160 according to a first discharge characteristic by providing the electrical energy from the ceramic storage capacitor 160 in accordance with a second discharge characteristic. As will be explained in more detail below, the second discharge characteristic is controlled by the energy conditioning circuit 164. In this manner, the temperature dependency and non -linear capacitance characteristics of the ceramic storage capacitor 160 can be accommodated, thus allowing ceramic storage capacitors to be used in the external defibrillator 100.

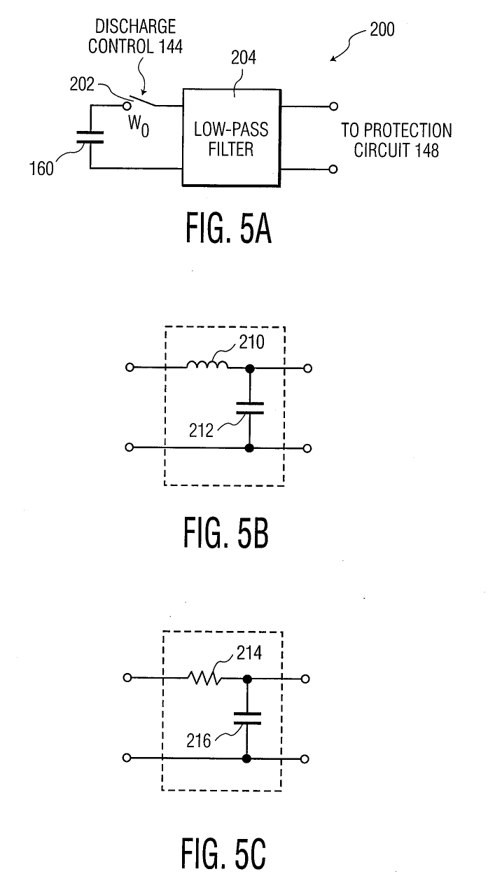

FIG. 5A illustrates an energy conditioning circuit 200 that can be used in the energy storage circuit 142. The energy conditioning circuit 200 utilizes a pulse modulation filtering scheme to deliver current-controlled or voltage-controlled discharge waveforms from the ceramic storage capacitor 160. The energy conditioning circuit 200 includes a switch 202 for pulse modulating the electrical energy discharging from the ceramic storage capacitor 160. The switch 202 couples and decouples the ceramic storage capacitor 160 at a relative high switching frequency ω₀ to form a series of pulses. The switching of the switch 202 can be controlled to provide pulses having various pulse widths and/or various pulse periods. An example of the switching frequency ω₀ of the switch 202 is approximately 30 kHz. However, alternative switching frequencies can also be used. The switching of the switch 202 is controlled by the discharge control circuit 144. As previously discussed, the discharge control circuit 144 controls the discharge of energy stored in the energy storage circuit 142. A low-pass filter 204 is coupled to the switch 202 to filter the waveform resulting from the relatively high -frequency switching of the switch 202. The output energy of the low-pass filter 204 is provided to the protection circuit 148, which in turn transfers the energy to the steering circuit 146, as previously described.

By filtering the energy pulses through the low -pass filter 104, the energy of the ceramic storage capacitor 160 discharging according to a first discharge characteristic can be delivered to the protection circuit according to a second discharge characteristic. The second discharge characteristic can be tailored by programming the discharge control circuit 144 to control the switch 202 to discharge the ceramic storage capacitor 160 through pulses of various widths and pulse periods, as previously mentioned. FIG. 5B illustrates a first low-pass filter network including an inductor 210 and a filtering capacitor 212 that can be used in the low-pass filter 204. FIG. 5C illustrates a second low-pass filter network including a resistor 214 and a filtering capacitor 216 that can also be used in the low-pass filter 204. Although FIG. 5A illustrates a particular energy conditioning circuit, alternative energy conditioning circuits can be utilized in other embodiments of the present invention as well.

What is claimed is:

1. An external defibrillator for providing a defibrillating pulse to a patient, comprising:
electrodes for coupling the defibrillating pulse to the patient;
a ceramic storage capacitor for storing electrical energy to be delivered as the defibrillating pulse, the ceramic storage capacitor having an electrical discharge characteristic according to which the stored electrical energy is discharged;
a charging circuit coupled to the ceramic storage capacitor and configured to electrically charge the ceramic storage capacitor;
an energy conditioning circuit coupled to the ceramic storage capacitor and configured to receive from the ceramic storage capacitor the electrical energy discharging according to the electrical discharge characteristic and in response provide the electrical energy discharging according to a modified electrical discharge characteristic; and
a steering circuit coupled to the energy conditioning circuit and the electrodes, the steering circuit configured to couple the electrical energy discharging according to the modified electrical discharge characteristic to the electrodes to deliver a defibrillating pulse having a defibrillating pulse characteristic to the patient.

2. The external defibrillator of claim 1 wherein the ceramic storage capacitor comprises a parallel plate ceramic capacitor having a first planar electrode, a second planar electrode, and a planar dielectric disposed between the first and second planar electrodes.

3. The external defibrillator of claim 2 wherein the planar ceramic capacitor has a generally rectangular shape.

4. The external defibrillator of claim 1 wherein the charging circuit comprises a flyback power supply.

5. The external defibrillator of claim 1 wherein the ceramic storage capacitor comprises a ceramic capacitor formed from compositions of at least one of lead, magnesium lanthanum, zirconium, and titanium.

6. The external defibrillator of claim 1 wherein the steering circuit comprises a steering circuit configured to deliver a biphasic defibrillating pulse to the patient.

7. An external defibrillator to generate a defibrillating pulse and deliver the same to a patient, the external defibrillator comprising:
an electrode having an electrode surface to electrically couple to the patient and further having a connector coupled to the electrode surface;
a high-voltage charging circuit for generating electrical energy;
an energy storage circuit coupled to the high-voltage charging circuit, the energy storage circuit having an output circuit to generate output energy having an output waveform in response to receiving input energy having an input waveform and further having a ceramic storage capacitor coupled to the output circuit and the high-voltage charging circuit to store the electrical energy and discharge the electrical energy as input energy; and
a switching circuit coupled to the energy storage circuit and the connector of the electrode, the switching circuit configured to couple the output energy of the output circuit to the patient to provide a defibrillating pulse through the electrode.

8. The external defibrillator of claim 7 wherein the ceramic storage capacitor of the energy storage circuit comprises a parallel plate ceramic capacitor having a first planar electrode, a second planar electrode, and a planar dielectric disposed between the first and second planar electrodes.

9. The external defibrillator of claim 8 wherein the planar ceramic capacitor has a generally rectangular shape.

10. The external defibrillator of claim 7 wherein the high-voltage charging circuit comprises a flyback power supply.

11. The external defibrillator of claim 7 wherein the ceramic storage capacitor of the energy storage circuit comprises a ceramic capacitor formed from compositions of at least one of lead, magnesium lanthanum, zirconium, and titanium.

12. The external defibrillator of claim 7 wherein the switching circuit comprises a switching circuit configured to deliver a biphasic defibrillating pulse to the patient.

13. A method for delivering a defibrillating pulse to a patient, comprising:
generating electrical energy;
storing the electrical energy in a ceramic capacitor;
discharging the electrical energy from the ceramic capacitor, the electrical energy discharging according to a first discharge characteristic;
conditioning the electrical energy discharging according to the first discharge characteristic to provide the electrical energy according to a second discharge characteristic; and
coupling the electrical energy discharging according to the second discharge characteristic to the patient to provide a defibrillating pulse having a defibrillating pulse characteristic.

14. The method of claim 13 wherein coupling the electrical energy discharging according to the second discharge characteristic to the patient to provide a defibrillating pulse having a defibrillating pulse characteristic comprises coupling the electrical energy to the patient to provide a biphasic defibrillating pulse.

15. The method of claim 13 wherein storing the electrical energy in a ceramic capacitor comprises storing electrical energy in a parallel plate ceramic capacitor having a first planar electrode, a second planar electrode, and a planar dielectric disposed between the first and second planar electrodes.

16. The method of claim 13 wherein storing the electrical energy in a ceramic capacitor comprises storing electrical energy in a ceramic capacitor formed from compositions of at least one of lead, magnesium lanthanum, zirconium, and titanium.
